(19) United States
(12) Patent Application Publication
(10) Pub. No.: US 2024/0392232 A1
(43) Pub. Date: Nov. 28, 2024

(54) STORAGE OF GRAM-NEGATIVE BACTERIA

(71) Applicant: Evonik Operations GmbH, Essen (DE)

(72) Inventors: Stefan GILCH, Dulmen (DE); Ina VIERHAUS, Velen (DE); Jennifer WIEGERS, Datteln (DE); Julia NIEWALDA, Selm (DE); Sabrina AOUAG, Essen (DE); Linda MICHEEL, Dorsten (DE); Eva-Maria ECKL, Marl (DE); Jochen KLEINEN, Heinsberg (DE)

(73) Assignee: Evonik Operations GmbH, Essen (DE)

(21) Appl. No.: 18/694,822

(22) PCT Filed: Sep. 22, 2022
(86) PCT No.: PCT/EP2022/074418

§ 371 Date: Mar. 22, 2024

(30) Foreign Application Priority Data
Sep. 23, 2021 (EP) 21198571.8

(51) Int. Cl. C12N 1/20; C12N 1/04; C12R 1/19; C12R 1/39; C12R 1/40

(52) U.S. Cl. CPC C12N 1/20; C12N 1/04; C12R 2001/19; C12R 2001/39; C12R 2001/40

(57) ABSTRACT

A method of producing a composition of Gram-negative bacteria includes
(a) subjecting a wet mass of Gram-negative bacteria to spray drying; and
(b) contacting the spray-dried Gram-negative bacteria from step (a) to a mixture including at least one hydrophobic, partially water-insoluble polyglycerol ester in combination with at least one emulsifier to form the composition of Gram-negative bacteria,
wherein the polyglycerol ester has a general formula (I) of,

MₐD_{b}T_{c} Formula (I)
wherein, M=[C₃H₅(OR)₂O_{1/2}],
   D=[C₃H₅(OR)₁O_{2/2}],
   T=[C₃H₅O_{3/2}],
   a=1 to 10;
   b=0 to 10;
   c=0 to 3;
      wherein, the sum total of a+b+c is 1 to 20,
      wherein the radicals R are independently selected from acyl radicals R′—C(=0)- and H, with the proviso that at least one radical R is not equal to H;
      wherein the radicals R′ are independently selected from monovalent aliphatic, saturated or unsaturated hydrocarbon radicals with 3 to 39 carbon atoms.

### FIELD OF THE INVENTION

The present invention relates a method of producing a liquid composition of Gram-negative bacteria, a method of improving storage stability of Gram-negative bacteria and a liquid composition of Gram-negative bacteria. In particular, the Gram-negative bacteria is first subjected to spray drying before contacting the spray-dried Gram-negative bacteria to a mixture comprising a polyglycerol ester and an emulsifier.

### BACKGROUND OF THE INVENTION

Gram-negative bacteria are known to have a high metabolic diversity and can thus be used in a variety of fields such as agriculture, personal care, health care and animal health. For example, the use of beneficial Gram-negative bacteria as alternatives to chemical pesticides and synthetic fertilizers in agricultural production is an area of increasing interest. Inoculant compositions comprising plant promoting bacteria or nitrogen fixing bacteria are well known and a commonly used biofertilizer. Rhizobacteria, a Gram-negative bacterium, are commonly applied as inoculants and include nitrogen-fixers and phosphate-solubilizers which enhance the availability of the macronutrients nitrogen and phosphorus to the host plant. The most commonly applied rhizobacteria are Rhizobium, Bradyrhizobium and closely related genera. Rhizobium and Bradyrhizobium are nitrogen-fixing bacteria that form symbiotic associations within nodules on the roots of legumes. Such behaviour increases host nitrogen nutrition and is important to the cultivation of soybeans, chickpeas and many other leguminous crops. Rhizobium also reduces the need of anthropogenic N-fertilization and thus lowers indirect CO2 emissions caused by the Haber-Bosch-Process.

However, since Gram-negative bacteria (in contrast to Gram-positive bacteria and/or fungi) cannot form persistence forms, so-called spores, and also only have a thin, single layer murein envelope to stabilize the cell membrane, the stability of Gram-negative bacteria is limited. In most of the areas of application however, a high level of stability both in storage and after use is required for customers to be convinced to use the Gram-negative bacteria and also to improve economic efficiency. In particular, bacterial inoculants are, only effective when, after application, the microorganisms are readily able to survive and thrive in soil conditions. Application of bacterial inoculant formulations and the soil environment itself subjects these inoculants to a variety of stresses, including temperature, mechanical, light, oxidative and osmotic stress, all of which impact the survivability of the bioactive. Thus, a further limitation to the use of bacterial inoculants is a low organism survival rate.

Many available products in the field of agricultural use have a shelf life based on their short-lived stability of only a few days to a few weeks maximum and have to be stored at low temperatures throughout this short period. Also, the products currently sold on the market are usually available as aqueous solutions or supported on peat or clay. All these products have a high-water activity (aW) that keeps the Gram-negative organisms metabolically active throughout their storage. Therefore, to prevent the bacteria from dying and to ensure high stability/durability of the Gram-negative bacteria during their storage period, an appropriate carbon source (usually sugar and/or biological extracts) is currently being added or the product containing the metabolically active Gram-negative bacteria must be stored at low temperatures in order to slow down the metabolic processes which makes the use of these Gram-negative bacteria costly and inconvenient.

Further, both chemical and biological active ingredients (pesticides, plant strengtheners, fertilizers) are often applied to the target site together with effect enhancers (additives, adjuvants). Dispersing additives, emulsifiers, defoamers etc. improve the physical properties of the product or concentrate. Spreading agents and adhesives ensure an improved effect or effectiveness of the chemical or biological active substances. However, since many effect enhancers only have a low level of biocompatibility, they can only be added in low concentrations (0.1 to 2%) as a tank mix. As a result, due to a lack of biocompatibility, it is often not possible to use the optimal concentration (about 40% or more adjuvant) of the enhancer in interaction with Gram-negative organisms in a single can. The effect enhancer and the Gram-negative cells are first brought into contact with one another in the tank. Furthermore, errors can be made by the user when the active substance and the effect enhancer are mixed manually in the tank, which lead to a partial reduction or a complete lack of effectiveness. Farmers also prefer in-can formulations because they only have to add one component (e.g. water), when the bacteria and adjuvant are already provided in the single can compared to individual additions of bacteria, adjuvant and water.

Accordingly, there remains a need in the art for a formulation of Gram-negative bacteria for storage that maintains the stability of the bacteria without jeopardizing on the efficiency of using the bacteria in the different fields. In particular, there is a need in the art for a formulation of Gram-negative bacteria with a durability/stability of at least 1-2 years at ambient temperature and increased humidity and a method for making these formulations of Gram-negative bacteria that provide improved bacterial yield and storage survivability.

### DESCRIPTION OF THE INVENTION

The present invention attempts to solve the problems above by providing an improved means of storage and transport of Gram-negative bacteria. In particular, any aspect of the present invention may be used to provide a liquid composition of spray-dried Gram negative bacteria as the main active ingredient mixed with at least one carrier which leads to improved handling and shelf life of the Gram negative bacteria compared to the prior art. In particular, an increased biological effectiveness of the Gram-negative bacteria active ingredient and/or bioavailability over a longer period may be achieved in comparison with the prior art. It has surprisingly been found that producing a liquid composition of Gram-negative bacteria using a method of first spray-drying the Gram-negative bacteria and then combining the dried cells with a mixture of polyglycerol ester and an emulsifier as a carrier for the cells may solve the problems above. In particular, the Gram-negative bacteria, the active ingredient and the mixture of polyglycerol ester and emulsifier, the activity enhancer, can be made up as a concentrate in a common premix and only have to be added to the tank in a single dose. Thus, allowing the use of an optimal concentration of the enhancer in interaction with the Gram-negative bacteria and also reducing chances of errors made by the user who usually mixes the Gram-negative bacteria with the activity enhancer in the tank manually as this step of manual mixing is skipped.

Further, the mixture of polyglycerol ester and emulsifier have a low water activity and thus growth of not only the Gram-negative bacteria is stopped but also that of unwanted growth of contaminating microorganisms, such as fungus and mould are prevented. This also helps increase the shelf-life of the Gram-negative cells.

According to one aspect of the present invention, there is provided a method of producing a liquid composition of Gram-negative bacteria, the method comprising
(a) subjecting a wet mass of Gram-negative bacteria to spray drying; and
(b) contacting the spray-dried Gram-negative bacteria from step (a) to a mixture comprising at least one hydrophobic, partially water-insoluble polyglycerol ester in combination with at least one emulsifier to form the liquid composition of Gram-negative bacteria,
   wherein the polyglycerol ester has a general formula (I) of,

MₐD_{b}T_{c} Formula (I)
wherein, M=[C₃H₅(OR)₂O_{1/2}],
   D=[C₃H₅(OR)₂O_{2/2}],
   T=[C₃H₅O_{3/2}],
   a=1 to 10, preferably 2 to 3, more preferably 2;
   b=0 to 10, preferably greater than 0 to 5, more preferably 1 to 3;
   c=0 to 3, preferably 0 to 1, more preferably 0;
      wherein, the sum total of a+b+c is 1 to 20, preferably 2 to 4, more preferably 3
      wherein the radicals R are each independently selected from the group consisting of acyl radicals R′—C(=0)- and H, with the proviso that at least one radical R is not equal to H;
      wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

The composition of Gram-negative bacteria may be a liquid formulation that protects the cells from penetrating water for a long time, which would lead to a decrease in stability. Spray-drying the Gram-negative bacteria and then combining the spray-dried Gram-negative bacteria with the mixture according to any aspect of the present invention leads to the desired increase in stability of the bacteria. Also, spray drying is able to reduce the aW in the cells from 1 to about 0.2-0.3 very quickly compared to other methods known in the art. Due to the biocompatibility of the mixture of hydrophobic, partially water-insoluble polyglycerol ester and emulsifier, the ratios of all components present are freely adjustable. The viscosity of the liquid composition of the Gram-negative bacteria, the final product also remains low in all mixing ratios. That enables the final product to be used up without any final product being wasted in the tank or spray containers, draining from the tanks and spray containers is also quick. Also, mixing is easy to handle and takes only little time. Further, no further emulsifiers have to be added outside of what is in the original mixture as, the mixture according to any aspect of the present invention is water-soluble. Addition of other emulsifiers and/or any other (bio-)active components may also have a toxic effect on the Gram-negative bacterial cells. Therefore, it is advantageous when such additions can be avoided.

The liquid composition of Gram-negative bacteria having a low aW offer the added benefit of improved stability and may eliminate the need for refrigeration due to dormant state of the cells. Such formulations also provide a lower risk of contamination.

It was surprisingly found that the spray drying process elevated the stability/viability of gram-negative cells.

Step (a) of spray-drying the Gram-negative bacteria, may be carried out in the presence of at least one additive. In particular, the Gram-negative bacterial cells may be stabilized during the dehydration process by introduction of at least one additive to the wet mass of the Gram-negative bacterial cells prior to subjecting the cells to spray-drying. The additive introduced to the wet mass of cells may be selected from at least one (i) silica, (ii) sugar with low water activity (aW value) and/or (iii) adhesive with low water activity (aW value). Any type of silica may be used according to any aspect of the present invention. In particular, silica may be used as a defined hydrophilic carrier with water-absorbing properties (maintenance of low water activity; aW). In one example, the silica used may be selected from the group consisting of Sipernat® 50, Sipernat® 50S, Sipernat® 22, Sipernat® 22S, Sipernat® 880, Zeolex® 7, Spherilex® 148, Zeolex® 23, and Zeolex® 23a. In particular, the silca used according to any aspect of the present invention may be Sipernat® 50, a carrier silica with high absorbency.

The (ii) sugar may be selected from the group consisting of isomalt, sucrose, isomaltulose, maltitol, erythritol and mannitol. In particular, the (ii) sugar may be isomalt. In one example, the (ii) sugar may be Risumalt® an isomalt. The (iii) adhesive with maintenance of low water activity used as a glass former may be selected from the group consisting of Gummi Arabicum, Xanthan and mica powder. In particular, the adhesive may be Gummi Arabicum. More in particular, the (ii) sugar may be Risumalt® and the (iii) adhesive may be Gummi Arabicum.

This step of spray drying is an essential part of the process, as rapid dehydration together with the vitrification of sugar (for example Risumalt®) is necessary for a high survival rate, stability, and shelf life of the Gram-negative bacteria.

The spray-drying may also be carried out in the presence of the medium in which in the Gram-negative cells were cultured. In particular, the medium may be Lysogeny broth (LB). More in particular, LB may be LB-Luria with 0.5 g/L NaCl. A skilled person would easily be able to prepare the LB medium used according to any aspect of the present invention.

In one example, the spray-drying according to any aspect of the present invention is carried out in the presence of a silica, a sugar and/or an adhesive. In particular, the spray-drying is carried out in the presence of a silica and a sugar. More in particular, the spray-drying is carried out in the presence of a silica and an isomalt. Even more in particular, the spray-drying is carried out in the presence of a silica, an isomalt and an adhesive. In particular, the spray-drying is carried out in the presence of a silica, an isomalt and Gummi Arabicum.

The spray-drying according to any aspect of the present invention is carried out in the presence of a silica, a sugar, an adhesive and/or LB. In particular, the spray-drying is carried out in the presence of a silica, a sugar, an adhesive and LB. More in particular, the spray-drying is carried out in the presence of a silica, an isomalt, an adhesive and LB. Even more in particular, the spray-drying is carried out in the presence of a silica, an isomalt, Gummi Arabicum and LB.

The spray-drying according to any aspect of the present invention may be carried out using any spray-dryer. In one example, a spray-dryer, Mini Spray Dryer B-290 (Buchi Labortechnik AG, Switzerland) may be used. In particular, the wet mass of Gram-negative bacteria may be spray-dried by a spray-dryer, whereby the spray-dried Gram-negative bacteria according to any aspect of the present invention may be obtained.

In step (a) according to any aspect of the present invention, the spray dryer may first be preheated with a very low fan speed. Spray drying may be achieved by allowing the inlet air temperature to be not so high so that the Gram-negative cells will survive the temperature. In particular, the inlet air temperature may be less than or equal to about 80° C., less than or equal to about 50° C., at about 30°−50° C. The spray drying may be with gas flow. The outlet air temperature may be less than or equal to about 55° C., less than or equal to about 54, or 50° C., at about 30°−55° C. The drying time may be considered to be proportional to the surface area of the silica and the control of water activity is inversely proportional to surface area of the silica. In one example, the spray-drying process of step (a) may be carried out using Buchi B-290 spray dryer with 10% pump power.

The composition of Gram-negative bacteria obtained after step (a) of spray drying has a water activity (aW) of about 0.01 to 0.5. The activity of water (aW) is a thermodynamic parameter. It is a measure of the amount of water available for chemical, biochemical and microbial reactions of samples, such as aqueous solutions and food, and can also be used to characterize the carrier (mixture) compositions. The water activity is given as a value and is defined as the ratio of the water vapour pressure above the sample (p) to the water vapour pressure of pure water (po) at the same temperature, a=p/po. The water activity corresponds to 1/100 of the relative equilibrium humidity (RGF). Equilibrium relative humidity is also referred to as equilibrium relative humidity (ERH). Pure water has a value of 1 and every addition of water-binding substances causes the value to drop below 1. In particular, the spray-dried Gram-negative bacteria obtained after step (a) of spray drying has a water activity (aW) of 0.01 to 0.45, 0.01 to 0.4, 0.01 to 0.35, 0.01 to 0.3, 0.01 to 0.25, 0.01 to 0.2, 0.05 to 0.5, 0.05 to 0.45, 0.05 to 0.4, 0.05 to 0.35, 0.05 to 0.3, 0.05 to 0.25, 0.05 to 0.2, 0.05 to 0.15, 0.05 to 0.1, 0.1 to 0.5, 0.1 to 0.45, 0.1 to 0.4, 0.1 to 0.35, 0.1 to 0.3, 0.1 to 0.25, 0.1 to 0.2, 0.1 to 0.15, 0.15 to 0.5, 0.15 to 0.45, 0.15 to 0.4, 0.15 to 0.3, 0.15 to 0.25, or 0.15 to 0.2. More in particular, the spray-dried Gram-negative bacteria obtained after step (a) of spray drying has a water activity (aW) of about 0.01, 0.05, 0.1, 0.15, 0.2, 0.25, 0.3, 0.35, or 0.4.

The spray-dried Gram-negative bacterial cells of step (a) may then be contacted with at least one mixture comprising at least one hydrophobic, partially water-insoluble polyglycerol ester in combination with at least one emulsifier to form the liquid composition of Gram-negative bacteria according to any aspect of the present invention. The mixture comprising at least one hydrophobic, partially water-insoluble polyglycerol ester in combination with at least one emulsifier may be considered an anhydrous additive that may be used as a carrier for the Gram-negative bacteria and leads to an increased shelf life/storage stability of the cells compared to water-dispersible powders or granules. The composition of Gram-negative bacteria according to any aspect of the present invention, a liquid formulation, may be diluted for example with water to form a liquid Gram-negative bacteria spray before being used in agriculture (i.e. Sprayed on crops and/or seeds). The mixture used in step (b) as carrier according to any aspect of the present invention shows very good adhesion of the drops of the Gram-negative bacteria onto the leaves of the crops. This carrier leads to good rain resistance of the Gram-negative bacteria on the plants. The Gram-negative bacteria are still present and effective on the leaves of the crops even after rain. The spray droplets of the liquid Gram-negative bacteria spray become larger and are therefore less prone to drifting off during the spraying process. The carrier also enables the spreading effect which equally covers/distributes the Gram-negative bacteria all over the surface of the target. In addition, the carrier can be produced sustainably from renewable raw materials and is also largely biodegradable. The carrier therefore shows a particularly good profile of properties.

The term ‘carrier’ refers to the mixture used according to any aspect of the present invention comprising at least one hydrophobic, at least partially water-insoluble polyglycerol ester in combination with at least one emulsifier. The carrier is able to transport the Gram-negative bacterial cells according to any aspect of the present invention to its final target (for example in agriculture to a part of the plant/crop). The carrier used according to any aspect of the present invention has also been disclosed in US 2017/0295782A.

The subjects according to the invention will be described hereinbelow without the invention being limited to these exemplary embodiments. If ranges, general formulae or classes of compounds are mentioned hereinbelow, they are intended to comprise not only the corresponding ranges or groups of compounds which are mentioned explicitly, but also all part-ranges and part-groups of compounds which can be obtained by selecting individual values (ranges) or compounds. When documents are cited for the purposes of the present description, the entire content of these is intended to be part of the disclosure of the present invention. When % data are provided hereinbelow, they are, unless otherwise specified, data in % by weight. In the case of compositions, the % data, unless otherwise specified, refer to the total composition. When averages are mentioned, they are, unless otherwise specified, mass averages (weight averages). When measured values are stated hereinafter, then these measured values, unless otherwise specified, were determined at a pressure of 101325 Pa and a temperature of 250° C.

The statement of a mass ratio of, for example, component (a) to component (b) of 0.1 means that a mixture comprising these two components includes 10% by weight of component (a) based on the total of the masses of components (a) and (b).

The phrase ‘partially water-insoluble’ means that the polyglycerol esters at a given temperature at a concentration of at least 0.01 g/I to 20 g/I in water already results in an opacity which can be discerned by the human eye, and preferably forms two phases at a concentration of at least 0.5 g/I to 2 g/I. The solubility may be determined at a temperature of below 80° C., particularly below 70, 60, 50, 40, 30, 29, 28, 27, 26, 25, 24, 23, 22, 21 and below 200° C. Furthermore, the solubility is particularly determined above 0° C., more particularly above 5, 10, 15, 16, 17, 18, 19, 20, 21, 22, 23, 24 and above 25° C. Even more particularly, the solubility may be determined at ambient temperature. In particular, the solubility may be determined at between 15° C. and 30° C., more particularly at from 20° C. to 25° C.

The HLB value (HLB stands for hydrophilic lipophilic balance), for example, can be used as a measure of the hydrophobicity. The polyglycerol ester according to any aspect of the present invention may have an HLB value of less than 8, particularly from 1 to 7, more particularly from 2 to 6.5. The HLB value may be at least 0.5, particularly at least 1, more particularly at least 2.

The HLB value can be determined using any method known in the art and is a recognized measure of the hydrophobicity. In particular, the HLB value may be determined by Griffin's method (I/I/. C. Griffin: Classification of surface-active agents by HLB, J. Soc. Cosmet. Chem. 1, 1949, pp. 311-326). The HLB value is calculated according to the formula

$$H L B = 20. ( 1 - \frac{m 1}{m} )$$

where m1 and m are the molar mass of the lipophilic portion (m1) and the total molecular mass (m) of the polyglycerol ester, respectively.

The molar masses are determined using any method known in the art. In one example, the molar mass may be determined by mass spectrometry, and the determination of the lipophilic portion may be done based on the mass-spectroscopy results applying stoichiometric rules which are well known to a skilled person.

In particular, the acyloxy radicals (also referred to as alkanoyloxy radicals) of the polyglycerol ester may comprise 4 to 40, preferably 8 to 22, in particular 10 to 18 carbon atoms.

More in particular, the polyglycerol ester according to any aspect of the present invention has a general formula (I) of,

MₐD_{b}T_{c} Formula (I)
wherein, M=[C₃H₅(OR)₂O_{1/2}],
   D=[C₃H₅(OR)₁O_{2/2}],
   T=[C₃H₅O_{3/2}],
   a=1 to 10, preferably 2 to 3, more preferably 2;
   b=0 to 10, preferably greater than 0 to 5, more preferably 1 to 3;
   c=0 to 3, preferably 0 to 1, more preferably 0;
      wherein, the sum total of a+b+c is 1 to 20, preferably 2 to 4, more preferably 3
      wherein the radicals R are each independently selected from the group consisting of acyl radicals R′—C(=0)- and H, with the proviso that at least one radical R is not equal to H;
      wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

In particular, at least one radical R corresponds to a radical of the formula R′—C(O)—. More in particular, M, D and T may be:

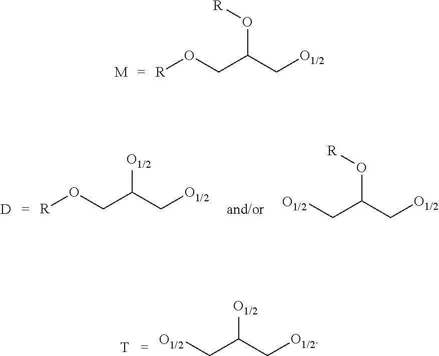

Even more in particular, the polyglycerol esters of the mixture according to any aspect of the present invention is of the Formula (II):

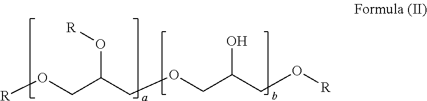

wherein
a=1 to 10, preferably 2 to 3, more in particular 2;
b=0 to 10, preferably greater than 0 to 5, more in particular 1 to 3;
with the proviso that:
a+b=2 to 20, preferably 2 to 4, especially 3;
wherein the radicals R are each independently selected from the group consisting of acyl radicals R′—C(=0)- and H, with the proviso that at least one radical R is not equal to H; where the radicals R′ are each independently selected from the group consisting of monovalent aliphatic, saturated or unsaturated hydrocarbon radicals with 3 to 39, preferably 7 to 21, in particular with 9 to 17 carbon atoms.

The polyglycerol esters of the mixtures according to any aspect of the present invention may have more than one radical R of the form R′—C(=0)-, particularly at least 2, more particularly at least 3.

The radicals R of the formula R′—C(O)— may be independent of each another identical or different acyl radicals of saturated or unsaturated fatty acids, where the fatty acids include 4 up to 40 carbon atoms, particularly, the fatty acids are selected from the group consisting of butyric acid (butanoic acid), caproic acid (hexanoic acid), caprylic acid (octanoic acid), capric acid (decanoic acid), lauric acid (dodecanoic acid), myristic acid (tetradecanoic acid), palmitic acid (hexadecanoic acid), stearic acid (octadecanoic acid), arachidic acid (eicosanoic acid), behenic acid (docosanoic acid), lignoceric acid (tetracosanoic acid), palmitoleic acid ((Z)-9-hexadecenoic acid), oleic acid ((Z)-9-hexadecenoic acid), elaidic acid ((E)-9-octadecenoic acid), cis-vaccenic acid ((Z)-11-octadecenoic acid), linoleic acid ((9Z,12Z)-9,12-octadecadienoic acid), alpha-linolenic acid ((9Z,12Z,15Z)-9,12,15-octadecatrienoic acid), gamma-linolenic acid ((6Z,9Z,12Z)-6,9,12-octadecatrienoic acid), di-homo-gamma-linolenic acid ((8Z,11Z,14Z)-8,11,14-eicosatrienoic acid), arachidonic acid ((5Z,8Z,11Z,14Z)-5,8,11,14-eicosatetraenoic acid), erucic acid ((Z)-13-docosenoic acid), nervonic acid ((Z)-15-tetracosenoic acid), ricinoleic acid, hydroxystearic acid, undecenoic acid, and mixtures thereof. In one example, the fatty acid may be a mixture of rapeseed oil acids, soya fatty acids, sunflower fatty acids, peanut fatty acids and tall oil fatty acids. In particular, for this context, the fatty acids may be radicals of oleic acid. When calculating the HLB value, the molar mass of the lipophilic molecule moiety is the arithmetic mean of the total of the molar masses of all of the radicals R′ which are present in the molecule. The total molar mass is calculated as defined hereinabove.

Sources of suitable fatty acids or fatty acid esters, especially glycerides, can be vegetable or animal fats, oils or waxes. For example, lard, beef tallow, goose fat, duck fat, chicken fat, horse fat, whale oil, fish oil, palm oil, olive oil, avocado oil, seed kernel oils, coconut oil, palm kernel oil, cocoa butter, cottonseed oil, pumpkin seed oil, maize seed oil, sunflower oil, wheat germ oil, grape seed oil, soybean oil, peanut oil, lupine oil, rapeseed oil, mustard oil, castor oil, jatropa oil, walnut oil, jojoba oil, lecithin, for example based on soy, rapeseed, or sunflower, bone oil, claw oil, borage oil, lanolin, emu oil, deer tallow, marmot oil, mink oil, safflower oil, hemp oil, pumpkin oil, evening primrose oil, tall oil, as well as carnauba wax, beeswax, candelilla wax, ouricuri wax, sugar cane wax, retamow wax caranday wax, raffia wax, esparto wax, alfalfa wax, bamboo wax, hemp wax, Douglas fir wax, cork wax, sisal wax, flax wax, cotton wax, dammar wax, tea wax, coffee wax, rice wax, oleander wax or wool wax may be sources of fatty acids or fatty acid esters.

In particular, the polyglycerol ester compounds have the formulas (I), or (II) with an arithmetic mean of 2.9 to 3.1 radicals of the form R′—C(=0) - and an HLB value of 4 to 6.5.

More in particular, the polyglycerol ester compounds have the formula (II) with the sum a+b being 3 and the arithmetic mean 2.9 to 3.1 radicals of the form R′—C(=0) - and an HLB-value of 4 to 6.5.

Even more in particular, the polyglycerol ester compounds may be of the formula (II) which have an arithmetic mean of 2.9 to 3.1 radicals of the form R′—C(=0) - and an HLB value of 4 to 6.5, where the acyl residues of fatty acid mixtures containing oleic acid, stearic acid, palmitic acid and gamma-linolenic acid, and said fatty acids particularly making up at least 85% by weight in the fatty acid mixture.

In one example, the polyglycerol ester compounds may be of the formula (II) which have an arithmetic mean of 2.9 to 3.1 radicals of the form R′—C(=0) - and an HLB value of 4 to 6.5, the acyl residues originate from fatty acid mixtures containing oleic acid, stearic acid, palmitic acid and gamma-linolenic acid, and said fatty acids particularly make up at least 85% by weight in the fatty acid mixture.

In another example, the polyglycerol ester compounds used according to any aspect of the present invention may be of the formula (II) which have an arithmetic mean of 2.9 to 3.1 radicals of the form R′—C(=0) - and an HLB value of 4 to 6.5, the mass fraction of oleic acyl residues is at least 75%, particularly 85%, more particularly 95% based on the mass of all acyl residues. Even more in particular, the polyglycerol ester is triglycerol trioleate.

The polyglycerol ester and the emulsifier of the mixture according to any aspect of the present invention may have a biodegradability of at least 50%, particularly at least 55%, more particularly at least 60%, with the maximum value of the biodegradability being 100%.

The emulsifier in the mixture according to any aspect of the present invention may be different from the polyglycerol ester. The emulsifier may be selected from the group consisting of fatty acid esters of polyhydric alcohols and their polyalkylene glycol derivatives, polyglycol derivatives of fatty acids and fatty alcohols, sorbitan fatty acid esters, ethoxylated and/or propoxylated sorbitan fatty acid esters, propoxylated sorbitan fatty acid esters, alkylphenol ethoxylates, propoxylates, alkylphenol ethoxylates, aminoxylated oxides, amine oxides, propoxylated amine oxides, aminoxylated amine oxides, aminoxylated propylene oxides, acetylenediol surfactants, ethoxylated and/or propoxylated acetylenediols, silicone surfactants and mixtures thereof. In particular, the emulsifier is selected from the group consisting of sorbitan fatty acid esters and ethoxylated sorbitan fatty acid esters. More in particular, the emulsifier is an ethoxylated sorbitan fatty acid ester or mixtures thereof.

The acyloxy radicals of the sorbitan fatty acid ester or ethoxylated sorbitan fatty acid ester have 4 to 40, in particular 8 to 22, more in particular 10 to 18 carbon atoms and/or that the sorbitan fatty acid ester or ethoxylated sorbitan fatty acid ester has 0 to 40, particularly 10 to 30, more particularly 15 to 25 oxyethylene groups. The fatty acids or fatty acid residues of the sorbitan fatty acid esters are particularly defined like the fatty acids or fatty acid residues of the polyglycerol esters. The acyl radicals (also referred to as alkanoyl radicals) are particularly derived from fatty acid mixtures containing oleic acid, stearic acid, palmitic acid and gamma-linolenic acid, said fatty acids particularly making up at least 85% by weight in the fatty acid mixture. Ethoxylated sorbitan fatty acid esters are particularly used, the mass fraction of oleic acid acyl residues being at least 75%, particularly 85%, more particularly 95%, based on the mass of all acyl residues.

The emulsifier according to any aspect of the present invention has an HLB value of greater than or equal to 9, particularly greater than or equal to 10, more particularly greater than or equal to 11. The HLB value may be a maximum of 16, more particularly a maximum of 15, even more particularly a maximum of 13. In particular, the emulsifier has an HLB value of 9 to 16, particularly 10 to 15, more particularly 11 to 13. The HLB value is determined as described above. The HLB value of the sorbitan fatty acid esters and/or ethoxylated sorbitan fatty acid esters is particularly determined as for the polyglycerol esters. The molar mass of the lipophilic part of the molecule results from the arithmetic mean of the sum of the molar masses of all radicals R′ present in the molecule as part of the acyl radicals R′—(CO)—. The radicals R′ are preferably as defined for the polyglycerol esters. The radical R′ as part of an acyl radical R′—(CO)— of the sorbitan fatty acid ester or ethoxylated sorbitan fatty acid ester is particularly selected from the group consisting of monovalent aliphatic, saturated or unsaturated hydrocarbon radicals with 3 to 39, preferably 7 to 21, particularly 9 to 17 carbon atoms. The calculation of the molar mass of the entire molecule is carried out as defined above. In particular, the emulsifier is polyethylene glycol-20-sorbitan trioleate. The number 20 indicates the average number of ethylene oxide units in the polyethylene glycol residue.

The HLB value of the polyglycerol ester and of the emulsifier are matched to one another. The polyglycerol ester has an HLB value of less than or equal to 8, particularly less than or equal to 7, more particularly less than or equal to 6.5, and the at least one emulsifier has an HLB value of greater than or equal to 9, particularly greater than or equal to 10, in particular greater than or equal to 11. The at least one polyglycerol ester has an HLB value of 0.5 to 8, particularly from 1 to 7, more particularly from 2 to 6.5 and the at least one emulsifier has an HLB value of 9 to 16, particularly from 10 to 15, more particularly from 11 to 13.

In particular, the at least one polyglycerol ester is triglycerol trioleate and the at least one emulsifier is polyethylene glycol 20 sorbitan trioleate. The combination of triglycerol trioleate and polyethylene glycol-20-sorbitan trioleate shows particularly advantageous properties as a carrier for a Gram-negative bacterium.

The mixture according to any aspect of the present invention consists to a predominant part of the at least one polyglycerol ester and, the at least one emulsifier. In particular, the mass fraction of the at least one polyglycerol ester together with the at least one emulsifier is at least 90%, particularly at least 95%, more particularly at least 99%, based on the total mass of the mixture. It is particularly advantageous if the mixture used as the carrier consists (essentially) of the at least one polyglycerol ester and the at least one emulsifier. It particularly applies that the mass fraction of the at least one polyglycerol ester based on the total mass of the mixture composition is 60% to 100%, particularly 70% to 90%, more particularly 75% to 85%; and the mass fraction of the at least one emulsifier based on the total mass of the mixture composition is 0% to 40%, particularly 10% to 30%, more particularly 15% to 25%. The mixture consists essentially of the at least one polyglycerol ester and the at least one emulsifier.

In particular, the mixture contains the at least one polyglycerol ester in a mass fraction based on the total mass of the carrier composition of 60% to 100%, particularly from 70% to 90%, more particularly from 75% to 85%, and that the mixture contains the at least one emulsifier which is at least one sorbitan fatty acid ester and/or at least one ethoxylated sorbitan fatty acid ester, particularly at least one ethoxylated sorbitan fatty acid ester, in a mass fraction based on the total mass of the mixture of 0% to 40%, particularly from 10% to 30%, more particularly from 15% to 25% contains.

More in particular, the mixture contains triglycerol trioleate as the polyglycerol ester in a mass fraction based on the total mass of the carrier composition from 60% to 100%, particularly from 70% to 90%, more particularly from 75% to 85%, and that the mixture contains polyethylene glycol 20 sorbitan trioleate as an emulsifier in a mass fraction based on the total mass of the mixture from 0% to 40%, particularly from 10% to 30%, more particularly from 15% to 25%.

The Gram-negative bacteria according to any aspect of the present invention may be from a species especially beneficial for agricultural applications, like N-fixating bacteria. In particular, Gram-negative bacteria according to any aspect of the present invention may be selected from the group consisting of Agrobacterium sp., Azispirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibactor sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp. More in particular, the Gram-negative bacteria may be selected from the group consisting of Acinetobacter baumani, Agrobacterium radiobacter, Agrobacterium tumefaciens, Azotobacter chroococcum, Azispirillum brasiliense, Bradyrhizobium arachidis, Bradyrhizobium japonicum, Bordetella pertussis, Brucella abortus, Brucella melitensis, Brucella suis, Burkholderia A39, Burkhulderia rinojensis, Campylobacter coli, Campylobacter foetus, Campylobacter jejuni, Chromobacterium subsugae, Curvibacter gracilis, Escherichia coli, Francisella tularensis, Haemophilus aphrophilus, Haemophilus haemolyticus, Haemophilus influenzae, Haemophilus parahaemolyticus, Haemophilus parainfluenza, Helicobacter pylori, Herbaspirillum hiltneri, Herbaspirillum lusitanum, Herbaspirillum rhizosphaerae, Klebsiella pneumoniae, Legionella pneumophilia, Luteibactor rhizovicina, Mesorihizobium cicero, Neisseria gonorrheae, Neisseria meningitidis, Pasteurella multocida, Pseudomonas aeruginosa, Pseudomonas azotoformans, Pseudomonas baetica, Pseudomonas brassicacearum, Pseudomonas brenneri, Pseudomonas cholororaphis, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas putida, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Rickettsia rickettsii, Salmonella typhimurium, Serratia plymuthica, Shigella boydii, Shigella dysenteriae, Shigella flexneri, Shigella sonnei, Sinorhizobium meliloti, Sphingomonas PDD-69b-4, Sphingomonas strain A3K041, Stenotrophomonas rhizophila, Variovorax ginsengisoli, Variovorax paradoxus, Vibrio cholerae, Vibrio opticus, Yersinia enterocolitica, Proteus mirabilis, Yersinia pestis, and Yersinia pseudotuberculosis.

Even more in particular, the Gram-negative bacteria is selected from the group consisting of Agrobacterium tumefaciens, Azotobacter chroococcum, Azispirillum brasiliense, Bradyrhizobium japonicum, Curvibacter gracilis, Herbaspirillum hiltneri, Herbaspirillum lusitanum, Herbaspirillum rhizosphaerae, Luteibactor rhizovicina, Pseudomonas aeruginosa, Pseudomonas azotoformans, Pseudomonas baetica, Pseudomonas brassicacearum, Pseudomonas brenneri, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Serratia plymuthica, Stenotrophomonas rhizophila, Variovorax ginsengisoli, and Variovorax paradoxus.

By first spray-drying the cells in step (a) according to any aspect of the present invention and reducing the water content of the cells, before contacting the cells with the mixture of the at least one polyglycerol ester and at least one emulsifier, the Gram-negative bacterial cells were found to have increased viability and thus also shelf life.

As a result of the synthesis, polyglycerol esters can contain residual amounts of water. It can therefore be advantageous to adjust the water content and thus the water activity, in particular to reduce it. This can be done, for example, by means of a thermal separation process. Thermal separation processes are known to the person skilled in the art under this term and include all processes which are based on the establishment of a thermodynamic phase equilibrium. In particular, thermal separation processes are selected from the group consisting of distillation, rectification, adsorption, crystallization, extraction, absorption, drying and freezing. More in particular, distillation and rectification may be used. Desiccants such as molecular sieves, e.g. Zeolites, or PharmaKeep (Mitsubishi Gas Chemical, Japan) can be used. In particular, the activity of water value of the mixture is less than 0.4, more in particular less than 0.3, even more in particular less than 0.25. Methods for determining the value are known to the person skilled in the art. The value is particularly determined as described in the examples in WO2020/234035.

The use of the mixture according to any aspect of the present invention leads to an increase in the storage stability of the microbiological active ingredient. The storage stability is determined as described in the examples in WO2020/234035.

According to another aspect of the present invention, there is provided a method for increasing the storage stability of at least one Gram-negative bacteria by
(a) subjecting a wet mass of Gram-negative bacteria to spray drying; and
(b) contacting the spray-dried Gram-negative bacteria from step (a) to a mixture comprising at least one hydrophobic, partially water-insoluble polyglycerol ester in combination with at least one emulsifier to form a liquid composition of Gram-negative bacteria,
   wherein the polyglycerol ester has a general formula (I) of,

MₐD_{b}T_{c} Formula (I)
wherein, M=[C₃H₅(OR)₂O_{1/2}],
   D=[C₃H₅(OR)₁O_{2/2}],
   T=[C₃H₅O_{3/2}],
   a=1 to 10, preferably 2 to 3, more preferably 2;
   b=0 to 10, preferably greater than 0 to 5, more preferably 1 to 3;
   c=0 to 3, preferably 0 to 1, more preferably 0;
wherein, the sum total of a+b+c is 1 to 20, preferably 2 to 4, more preferably 3
wherein the radicals R are each independently selected from the group consisting of acyl radicals R′—C(=0)- and H, with the proviso that at least one radical R is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

According to a further aspect of the present invention, there is provided a liquid composition of Gram-negative bacteria comprising
a spray-dried mass of Gram-negative bacteria with an activity of water of 0.05 to 0.4;
a silica,
a sugar; and
a mixture comprising at least one hydrophobic, partially water-insoluble polyglycerol ester in combination with at least one emulsifier
wherein the polyglycerol ester has a general formula (I) of,

MₐD_{b}T_{c} Formula (I)
wherein, M=[C₃H₅(OR)₂O_{1/2}],
   D=[C₃H₅(OR)₁O_{2/2}],
   T=[C₃H₅O_{3/2}],
   a=1 to 10, preferably 2 to 3, more preferably 2;
   b=0 to 10, preferably greater than 0 to 5, more preferably 1 to 3;
   c=0 to 3, preferably 0 to 1, more preferably 0;
wherein, the sum total of a+b+c is 1 to 20, preferably 2 to 4, more preferably 3
wherein the radicals R are each independently selected from the group consisting of acyl radicals R′—C(=0) - and H, with the proviso that at least one radical R is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

In particular, the sugar according to any aspect of the present invention may be an isomalt. The presence of the sugar and/or silica maintains the low aW of the final product, the liquid composition of the Gram-negative bacteria. Further, the presence of silica has the added benefit of being able to absorb water, has good handling properties/flowability making it suitable and easy to use. Further, silica increases the volume of the final product making the handling of the final product also easier and more convenient. Similarly, the presence of sugar in the final product forms a kind of covering or layer over the Gram-negative bacteria by glassification (possibly forming a quasi “glass coating”) to possibly reduce water influx and maybe also acting as outgrowth agent for the cells after the storage.

A varied concentration of silica and/or sugar may be present in the liquid composition of Gram-negative bacteria. WO2020104612A1 shows a range of concentrations of silica and/or sugar that may be applicable according to any aspect of the present invention. In one example, the percentage of silica may be about 6,71% (g/g) and the percentage of sugar may be about 15.91% (g/g).

In particular, according to any aspect of the present invention, the emulsifier may be a sorbitan fatty acid ester or an ethoxylated sorbitan fatty acid ester.

### BRIEF DESCRIPTION OF THE FIGURES

FIG. 1 are graphs showing the stabilization of Pseudomonas fluorescens using the method according to any aspect of the present invention. FIG. 1(A) are the survival rate and storage results of direct liquid formulation and FIGS. 1(B) and (C) are the survival rate and storage results of spray dried Pseudomonas fluorescens then formulated in liquid 1:10 adjuvants.

FIG. 2 are graphs showing the stabilization of Azospirillum brasilense using the method according to any aspect of the present invention. FIGS. 2(A) and B are the survival rate and storage results of direct liquid formulation and FIG. 2(C) is the survival rate and storage results of spray dried Azospirillum brasilense then formulated in liquid 1:10 adjuvants.

FIG. 3 shows the stabilization of Azotobacter chroococcum using the direct liquid formulation.

FIG. 4 are graphs showing the stabilization of Bradyrhizobium japonicum using the method according to any aspect of the present invention. FIG. 4(A) is the survival rate and storage results of direct liquid formulation and FIG. 4(B) is the survival rate and storage results of spray dried Bradyrhizobium japonicum then formulated in liquid 1:10 adjuvants.

FIG. 5 are graphs showing the stabilization of Escherichia coli using the method according to any aspect of the present invention. FIG. 5(A) is the survival rate and storage results of direct liquid formulation and FIG. 5(B) is the survival rate and storage results of spray dried Escherichia coli then formulated in liquid 1:10 adjuvants.

FIG. 6 are graphs showing the stabilization of Pseudomonas putida using the method according to any aspect of the present invention. FIG. 6(A) is the survival rate and storage results of direct liquid formulation and FIG. 6(B) is the survival rate and storage results of spray dried Pseudomonas putida then formulated in liquid 1:10 adjuvants.

FIG. 7 is a graph showing the stability of the adjuvants in combination with Pseudomonas fluorescens when the cells are pretreated differentially (spray dried or not).

FIG. 8 is a graph showing the stability of the adjuvants in combination with Pseudomonas fluorescens when the cells are pretreated differentially (spray dried or not).

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Materials and Methods

Devices used are shown in Table 1.

TABLE 1
Lab equipment used for cultivation, spray drying and
analyzing biomass in the described experiments.

| Equipment | Provider |
|---|---|
| DASGIP 1L vessels | Eppendorf Vertrieb Deutschland |
| | GmbH, Germany |
| Infors shaker Multitron Pro | Infors GmbH, Germany |
| Revolver rotator D-6050 | neoLab Migge GmbH, Germany |
| Balance Kern EW 12000 | Kern & Sohn GmbH, Germany |
| Magnetic Stirrer | IKA GmbH & Co. KG, Germany |
| Minishaker M52 | IKA GmbH & Co. KG, Germany |
| Pipettes (Research/ Reference) | Eppendorf Vertrieb Deutschland |
| | GmbH, Germany |
| Mini Spray Dryer B-290 | Büchi Labortechnik AG, Switzerland |
| Centrifuge Sigma 4K15 | Sigma Laborzentrifugen GmbH, |
| | Germany |
| Photometer Genesysy 10S UV-VIS | Thermo Scienfic, Germany |
| Labmaster -Aw Neo | Novasina AG, Switzerland |
| Moisture Analyzer, classic plus | Mettler Toledo |
| Climate cabinet | Binder GmbH, Germany |

Materials used for formulation experiments of freshly grown biomass are shown in Table 2.

TABLE 2
Polyethers and alternative compounds used to formulate
biomass fir starage and agricultural application.

| Material | Supplier |
|---|---|
| Polyether SP133 | Evonik Operations GmbH |
| NaCl | Carl Roth GmbH + Co. KG |
| Glycerol | Alfa Aeser |
| PEG400 | Carl Roth GmbH + Co. KG |

BREAK-THRU® SP 133 from Evonik, a mixture of 80% by weight triglycerol trioleate and 20% by weight polyethylene glycol 20 sorbitan trioleate, was used as the carrier composition/mixture according to any aspect of the present invention.

### Example 1

### Cell Cultivation

To always generate reliable results using cells it is very important to have a well-controlled cultivation method yielding always the same cell quality. We use the DASGIP-parallel-fermentation-system and the same fermentation protocol and -program for every cell production process. Depending on the microorganism to be cultivated an appropriate medium had to be chosen.

The Media are shown in Table 3.

TABLE 3
Media used to cultivate different microorganisms
for formulation trials.

| Organism | Medium | Temperature |
|---|---|---|
| Pseudomonas fluorescens | M1Ps | 30° C. |
| Pseudomonas putida | M1Ps | 30° C. |
| Escherichia coli | M1Es | 30° C. |
| Azospirillum brasilense | M1Az | 32° C. |
| Bradyrhizobium japonicum | M1Br | 28° C. |

### M1Ps

| | | |
|---|---|---|
| (NH₄)₂SO₄ | 2.20 | g/kg |
| NaCl | 0.02 | g/kg |
| MgSO₄ × 7 H₂O | 4.00 | g/kg |
| CaCl₂ × 2 H₂O | 0.10 | g/kg |
| KH₂PO₄ | 6.00 | g/kg |
| Glucose | 15.0 | g/kg |
| Trace elements TE-Ps | 30 | L/kg |

### TE-Ps

| | | |
|---|---|---|
| ZnSO₄ × 7 H₂O | 0.200 | g/kg |
| MnSO₄ × H₂O | 0.090 | g/kg |
| Na₃Citrate × 2 H₂O | 2.000 | g/kg |
| CuSO₄ × 5 H₂O | 0.100 | g/kg |
| NiSO₄ × 6 H₂O | 0.002 | g/kg |
| Na₂MoO₄ × 2 H₂O | 0.003 | g/kg |
| H₃BO₃ | 0.030 | g/kg |
| FeSO₄ × 7 H₂O | 4.000 | g/L |

Dissolve in water to reach 1 kg and filter-sterilize.

### M1Es

| | | |
|---|---|---|
| KH₂PO₄ | 12.07 | g/kg |
| Na₃Citrate × 2 H₂O | 2.240 | g/kg |
| (NH₄)₂SO₄ | 4.300 | g/kg |
| MgSO₄ × 7 H₂O | 3.000 | g/kg |
| Yeast extract | 1.500 | g/kg |
| (NH₃) Fe(III) Citrate | 0.170 | g/kg |
| Glucose | 15.00 | g/kg |
| Trace elements TE-Es | 5.000 | mL/kg |

### TE-Es

| | | |
|---|---|---|
| HCL 37% | 80 | mL/L |
| MnCl₂ × 4 H₂O | 1.90 | g/L |
| ZnSO₄ × 7 H₂O | 1.90 | g/L |
| NaEDTA × 2 H₂O (Titriplex III) | 0.90 | g(L |
| H₃BO₃ | 0.30 | g/L |
| Na₂MoO₄ × 2 H₂O | 0.30 | g/L |
| FeSO₄ × 7 H₂O | 17.8 | g/L |

| | |
|---|---|
| CuCl₂ × 2 H₂O | 0.20/L |

### M1Az

| | | |
|---|---|---|
| KH₂PO₄ | 4.000 | g/L |
| K₂HPO₄ | 4.580 | g/L |
| Yeast extract | 5.000 | g/L |
| MgSO₄ × 7 H₂O | 0.500 | g/L |
| NaCl | 0.100 | g/L |
| CaCl₂ × 2 H₂O | 0.015 | g/L |
| Na₂MoO₄ × 2 H₂O | 0.002 | g/L |
| FeCl₃ | 0.010 | g/L |

| | |
|---|---|
| Fructose | 10.0 /L |

### M1Br

| | | |
|---|---|---|
| NaH₂PO₄ × 2 H₂O | 0.157 | g/L |
| MgSO₄ × 7 H₂O | 0.180 | g/L |
| Na₂SO₄ | 0.250 | g/L |
| NH₄Cl | 0.320 | g/L |
| CaCl₂ × 2 H₂O | 0.013 | g/L |
| Yeast extract | 0.250 | g/L |
| HEPES | 1.300 | g/L |
| MOPS | 1.100 | g/L |
| FeCl₃ | 0.004 | g/L |
| Mannitol | 20.00 | g/L |

All of the aforementioned organisms were cultured firstly from a plate/cryo stock in medium (25 ml in a 250 ml baffled shake flask) at 28-32° C. and 200 rpm for about 20 h as the first tier preculture. From this culture 50 ml broth in a 500 ml shake flask was inoculated in a way that this second tier preculture started at an OD₆₀₀ of 0.2. This shake flask was incubated at 28-32° C., 200 rpm for about 7h to reach an OD₆₀₀ of ˜3-7. In order to inoculate the reactors with an optical density of 0.7, the OD₆₀₀ of the second preculture stage was measured and the amount of culture required to act as inoculum was calculated.

The starting volume in the reactor was 300 mL, which was tempered to the desired temperature with adjusted pH and dissolved oxygen (DO). The system was programmed to keep a DO of 30% by adjusting stirrer speed first and air flow second. Detailed controller parameters are shown in Table 4. The pH value was kept constant at the desired pH value by feeding 12.5% ammonia solution or 2.5 M H₂SO₄, controlled by the DASGIP Control software. Growth conditions for different microorganisms are shown in Table 3.

After inoculation cells were grown to an optical density of OD₆₀₀˜15-20. These values are reached in the batch phase without any substrate limitation applied to the cells in less than 20 h time.

After the cells grew to the desired optical density a sample of 25 mL was taken and stored for direct-liquid-formulation experiments. The residual majority of the cells was harvested by centrifugation: cells were transferred aseptically to centrifuge buckets, the buckets tared, and cells pelleted by centrifugation for 20 min at 12 000 g. Residual moistness was measured using the Mettler-Toledo “Moisture Analyzer, classic plus” for 2 h at 90° C. This measurement gave information about the cell dry weight corresponding to the cell wet weight of the cell pellet. These data are important for the correct composition of the spray draying solution.

TABLE 4
Controller parameters for cell cultivation in a 1L DASGIP
reactor using 300 mL starting volume in a batch process.

Dissolved oxygen

| | |
|---|---|
| (DO) regulator | pH regulator |

| | | | | |
|---|---|---|---|---|
| Preset | 0% | Preset | 0 ml/h | T regulator |

| P | 0.1 | P | 5 | | P | 6K |
|---|---|---|---|---|---|---|
| Ti | 300 s | Ti | 200 | s | Ti | 8520 s |
| min | 0% | min | 0 | ml/h | | |
| max | 100% | max | 40 | ml/h | | |

| N (Rotation) | from | to | XO2 (gas mixture) | from | to |
|---|---|---|---|---|---|
| growth and | 0% | 30% | growth and | 0% | 100% |
| biotrans- | 400 rpm | 1500 rpm | biotrans- | 21% | 21% |
| formation | | | formation | | |

| F (gas flow rate) | from | to |
|---|---|---|
| growth and | 15% | 80% |
| biotrans- | 6 sL/h | 72 sL/h |
| formation | | |

### Example 2

### Spray Drying

For spray drying a feed solution had to be prepared which contained the cells and several additives found to stabilize the cells during the dehydration process. Sipernat 50, a silica carrier material, risumalt and arabic gum (gummi arabicum) as glass former were also needed as Luria Broth medium as nutrient supplement for the cells. The detailed composition of the spray drying feed solution is shown in Table 5. A premix was prepared from NaCl solution, gummi arabicum, Risumalt and Sipernate by mixing the NaCl solution and gummi arabicum, stirring for 1 h, adding risumalt and stirring for one more hour, adding the Sipernat 50® and pasteurize the mixture tor 2 h at 80° C. This Premix can be stored a few weeks for later use in different spray drying experiments. After adding the cells and LB medium a sample was withdrawn for CFU analysis.

TABLE 5
Composition ot spray drying feed solution.

| | [g] |
|---|---|
| 0.9% NaCl-solution | 35.61 |
| Gummi Arabicum | 5.71 |
| Risumalt | 15.97 |
| Sipernat 50 ® | 6.71 |
| Luria Broth | 18.00 |
| Cells (wet weight) | 18.00 |

NaCl solution was mixed with gummi arabicum by stirring for 1 h at room temperature. Risumalt® was added afterwards and again mixed by stirring for 1 h at room temperature, Sipernat® was added last and this premix pasteurized for 2 h at 80° C. This premix was prepared as stock solution before starting the experiment. Cells and LB medium were added after cooling down the premix and stirred again for 1 h at room temperature.

For the dehydration process itself the Buchi “Mini Spray Dryer B-290” was used. After integration of the device, the system was sterilized at 180° C. for 1 h. Afterwards pre-run was carried out by spraying water into the drying chamber using the parameters shown in Table 6, all according to the manufacturers manual. During this pre-run the mist jet was focused into the drying chamber.

After these preparations the spray drying solution containing the cells was fed to the system. This procedure lasted for about 15 min. Applied parameters are shown in Table 7. In the spray dryer the cells were dehydrated very quickly. They pass the hot zone in such a short period of time, that they were not harmed by the heat very much. The sugars mixed into the spray drying solution formed a glass surrounding the cells, shielded them from the air and stabilized their proteins and DNA.

TABLE 6
Spray draying pre-run using pure water to
focus the mist jet into the drying chamber.

| Parameters | |
|---|---|
| Aspirator [%] | 100 |
| Q-Flow [sL/h] | 60 |
| Pump rate [%] | 12-17 |
| Outlet-Temperature [° C.] | 53 |
| Inlet-Temperature [° C.] | 80 |

TABLE 7
Spray drying parameters for the cell containing solution.

| Parameters | |
|---|---|
| Aspirator [%] | 100 |
| Q-Flow [sL/h] | 60 |
| Outlet-Temperature [° C.] | 53 |
| Inlet-Temperature [° C.] | 80 |
| Pump Power [%] | 17 |
| Throughput | 100 g / 15 min |
| Water flush | yes |
| Air flush | yes |

The dehydrated material was collected by the glass cyclone and fell into the collecting vessel. This cell dust was used for the formulation trials. First of all, water activity of the spray dried material was measured using the “Labmaster-Aw Neo” produced by Novasina AG, Switzerland. The water activity should be <0.3.

### Example 3

### Formulation Experiments —Dry Matter Formulation (DMF)

For the DMF trials spray dried material was mixed with the formulation agents as described below in the comparative example 4 in the ratios shown in Table 8.

TABLE 8
Composition of the dry-matter-formulation trials.

| | | Amount of | Amount of |
|---|---|---|---|
| Agent | Ratio agent/dry matter | agent | dry matter |
| 0.9% NaCl | 90% | 9 mL | 1 g |
| SP133 (as disclosed | 90% | 9 mL | 1 g |
| in Example 1) | | | |
| Glycerin | 90% | 9 mL | 1 g |
| PEG400 | 90% | 9 mL | 1 g |

As described for the Direct Liquid Formulation (DLF) below, samples were taken directly after mixing the spray dried material with the formulation agents and after certain periods of storage time. These samples were subjected to CFU analysis together with the sample of the spray drying solution withdrawn before the dehydration process in the spray dryer.

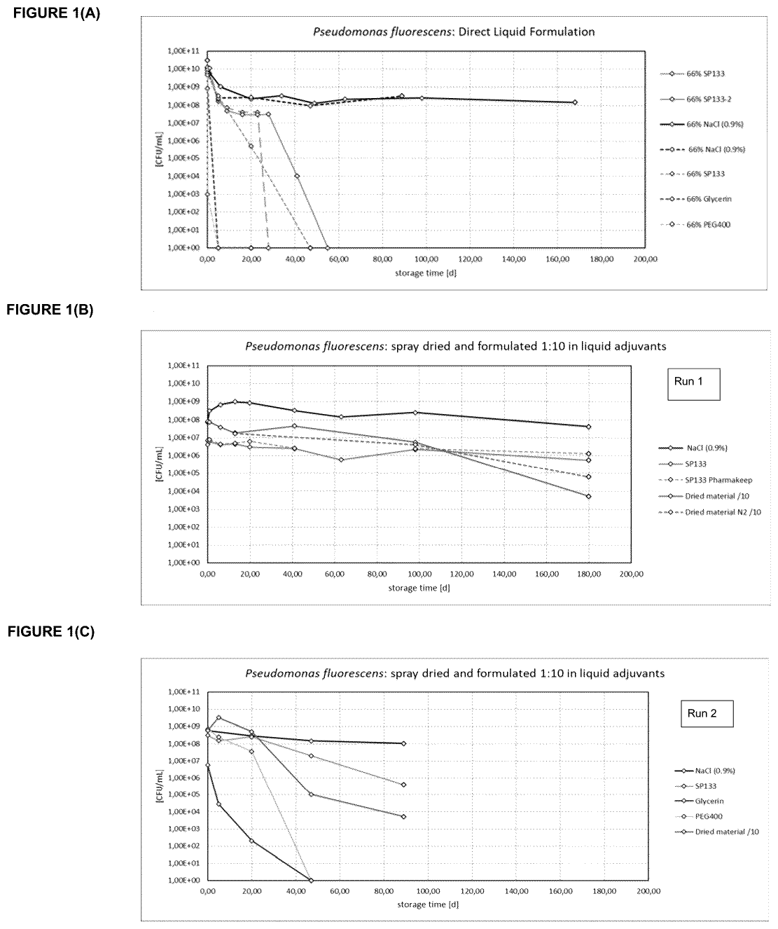

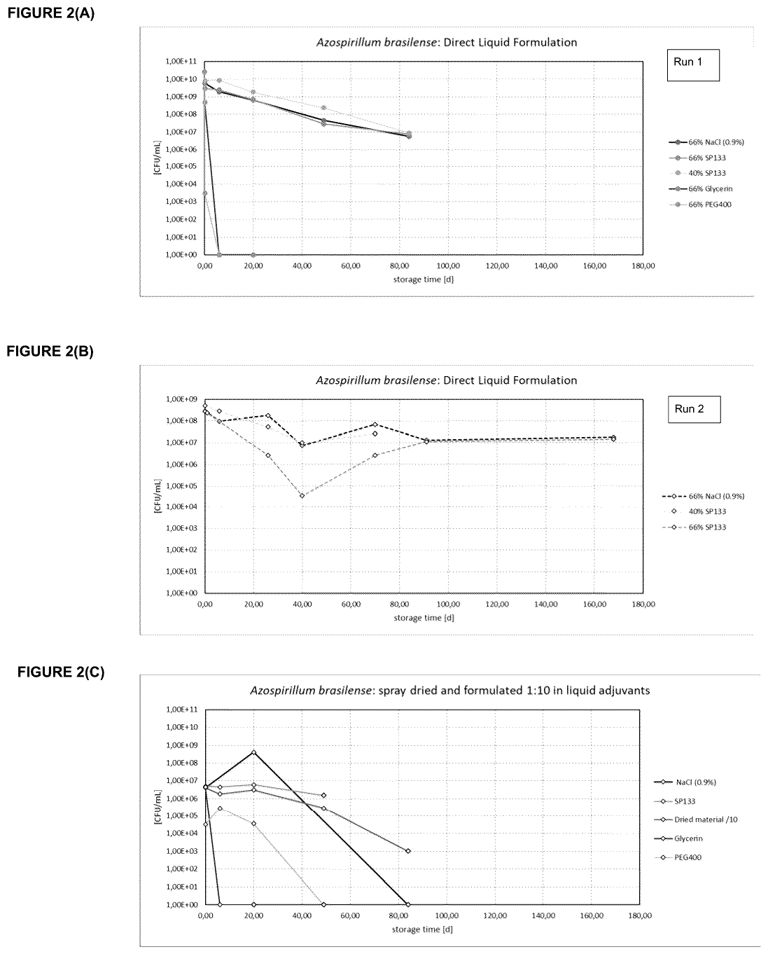

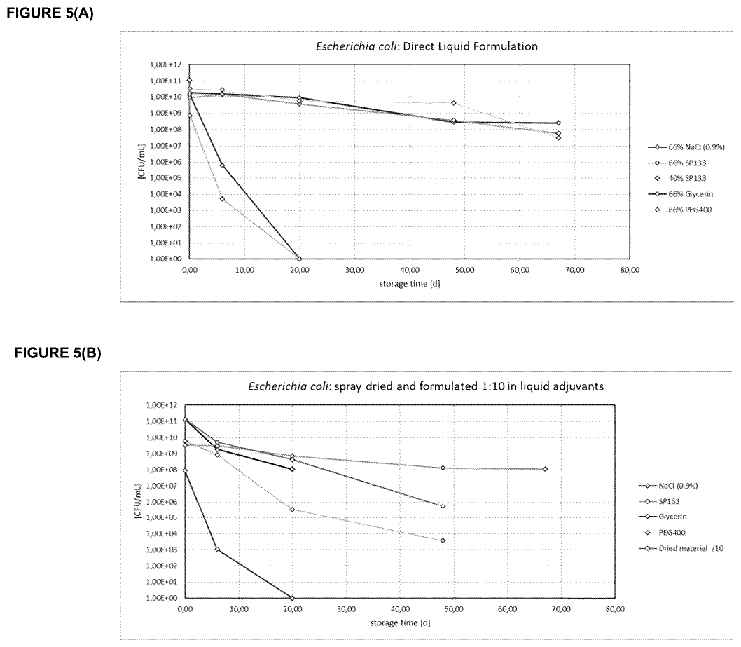

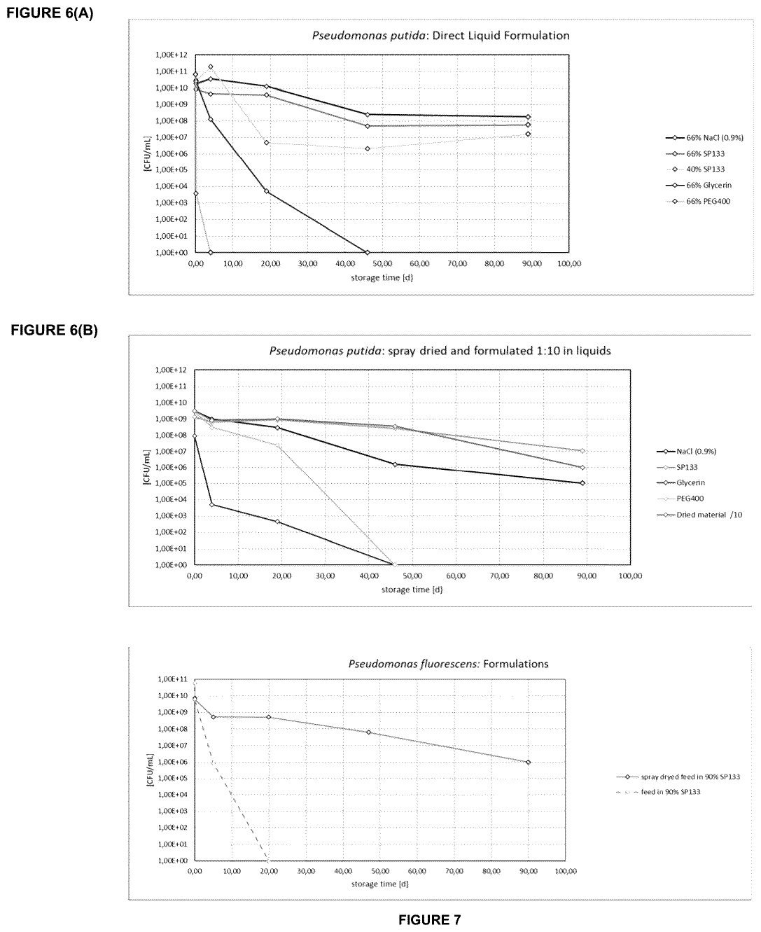

The results are shown in FIGS. 1B and C for Pseudomonas fluorescens, FIG. 2C for Azospirillum brasilense, FIG. 4B for Bradyrhizobium japonicum, FIG. 5B for Escherichia coli and FIG. 6B for Pseudomonas putida. After spray drying, the AW of the spray dried material of Pseudomonas fluorescens, was 0.1589 and the survival rate after spray drying was 0.305% in Run 1, the AW of the spray dried material of Pseudomonas fluorescens, was 0.2283 (residual moisture was 6.29%) and the survival rate after spray drying was 2.69% in Run 2. The residual moisture content was measured in every case in the examples with the Mettler Toledo Moisture Analyzer HB43-S (Germany) following the user manual. The weight of the sample was measured in the chamber at t=0. Then the sample was heated up by a halogen lamp, and the weight was continually measured, until a plateau was reached. The mass difference between t=0 and the plateau was the residual moisture content.

The AW of the spray dried material of Azospirillum brasilense, was 0.1820 (residual moisture 6.04%) and the survival rate after spray drying was 0.02%. In contrast the AW of spray dried material 1:10 in PEG was measured to be 0.2738 and after 52 days of storage, the AW was 0.3903 as shown in FIG. 2C.

According to FIG. 4B, the AW of the spray dried material of Bradyrhizobium japonicum, was 0.3442 and the survival rate after spray drying was 46.6%. As shown in FIG. 5B, the AW of the spray dried material of Escherichia coli, was 0.2233 (residual moisture 7.8%). According to FIG. 6BB, the AW of the spray dried material of Pseudomonas putida, was 0.1673 (residual moisture 5.58%) and the survival rate after spray drying was 10.35%.

As a control, all components used for spray drying (Table 5) were mixed, 90% SP133 was added and subsequently stored at room temperature but without applying the spray drying process itself. The mixture reveals an Aw value of about 0.65 and the activity (CFU) of Pseudomonas fluorescens decreased rapidly upon storage at room temperature. Consequently, this is not sufficient to deliver high stability/survival rates to the cell. In contrast, when the same components (Table 5) are mixed, spray dried (survival rate: 2.69%) and subsequently mixed with 90% SP133 the Aw of the product was found to be 0.2283 (residual moisture 6.29%) and stability of the spray dried material in 90% SP133 is way higher than compared to the non-spray dried product.

Since dry material is not formulated in liquid adjuvant like described in the headline of the graph, the term x/10x was added.

### Example 4 (Comparative Example)

### Formulation Experiments—Direct Liquid Formulation (DLF)

The sample of fermentation broth taken shortly before harvesting the cells was now used to prepare the first formulations: Broth was first centrifuged and the biomass pallet (33%) mixed with the different agents (66%) selected to be tested for their cell stabilizing properties as shown in Table 9. Mixing was achieved by vortexing for 10 sec. If this was not enough manual stirring using an inoculation loop was applied. Immediately after mixing a sample of each formulation was taken and used for CFU analysis to determine the start vitality of the cells.

TABLE 9
Composition of the direct-liquid-formulation trials.

| Agent | Ratio agent/broth | Amount of agent | | Amount of broth |
|---|---|---|---|---|
| 0.9% NaCl | 66% | 6 | mL | 3 ml |
| SP133 | 66% | 6 | g | 3 ml |
| SP133 | 40% | 4 | g | 6 ml |
| Glycerin | 66% | 6 | g | 3 ml |
| PEG400 | 66% | 6 | g | 3 ml |

The formulations were than incubated in a “Revolver Rotater”, produced by neoLab Migge GmbH, Germany at 20° C. Further samples were taken after certain storage periods to observe the vitality pattern over time. Those samples had to be taken aseptically always. They all were subjected to CFU analysis by plating dilutions prepared in LB medium and colony counting after incubation at 28-32° C. overnight.

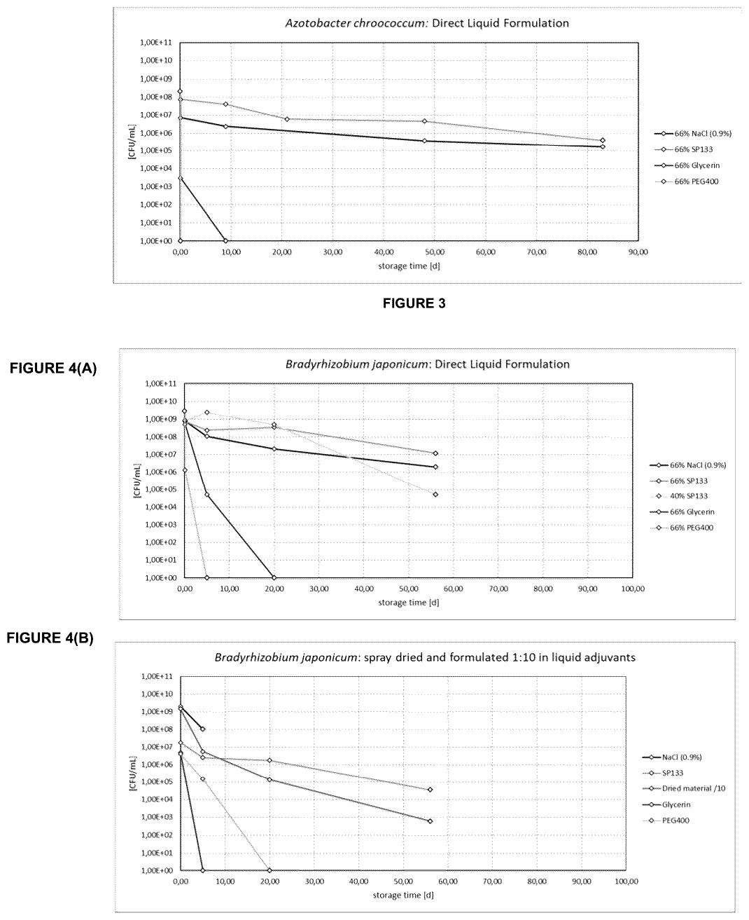

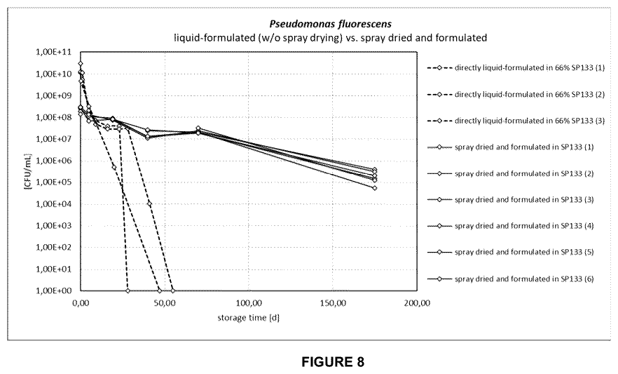

The results are shown in FIGS. 1A, 7 and 8 for Pseudomonas fluorescens, FIGS. 2A and B for Azospirillum brasilense, FIG. 3 for Azotobacter chroococcum, FIG. 4A for Bradyrhizobium japonicum, FIG. 5A for Escherichia coli and FIG. 6A for Pseudomonas putida. Experiments have been repeated 3 times for liquid formulation (without spray-drying) and 6 times for the formulation including the spray-drying process of Pseudomonas fluorescens in FIG. 8.

1. A method of producing a liquid composition of Gram-negative bacteria, the method comprising
(a) subjecting a wet mass of Gram-negative bacteria to spray drying; and
(b) contacting the spray-dried Gram-negative bacteria from step (a) to a mixture comprising at least one hydrophobic, partially water-insoluble polyglycerol ester in combination with at least one emulsifier to form the liquid composition of Gram-negative bacteria,
wherein the polyglycerol ester has a general formula (I) of,
MₐD_{b}T_{c} Formula (I)
wherein, M=[C₃H₅(OR)₂O_{1/2}],
D=[C₃H₅(OR)₁O_{2/2}],
T=[C₃H_{503/2],}
a=1 to 10, preferably 2 to 3, more preferably 2;
b=0 to 10, preferably greater than 0 to 5, more preferably 1 to 3;
c=0 to 3, preferably 0 to 1, more preferably 0;
wherein, the sum total of a+b+c is 1 to 20, preferably 2 to 4, more preferably 3
wherein the radicals R are each independently selected from the group consisting of acyl radicals R′—C(=0)- and H, with the proviso that at least one radical R is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

2. The method according to claim 1, wherein the mixture comprises about 80% of the polyglycerol ester and about 20% of the emulsifier.

3. The method according to claim 1, wherein the emulsifier is a sorbitan fatty acid ester or an ethoxylated sorbitan fatty acid ester, preferably an ethoxylated sorbitan trioleate.

4. The method according to claim 1, wherein the emulsifier is a polyethylene glycol-20 sorbitan trioleate.

5. The method according to claim 1, wherein the spray drying in step (a) is carried out in the presence of a silica, an isomalt and/or gum arabic.

6. The method according to claim 1, wherein the spray-dried Gram-negative bacteria after step (a) has a water activity of 0.05 to 0.4.

7. The method according to claim 1, wherein the spray-dried Gram-negative bacteria after step (a) has a water activity of about 0.4.

8. The method according to claim 1, wherein the Gram-negative bacteria is selected from the group consisting of Gram-negative bacteria selected from the group consisting of Agrobacterium sp., Azispirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibactor sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp.

9. The method according to claim 1, wherein the Gram-negative bacteria is selected from the group consisting of Acinetobacter baumani, Agrobacterium radiobacter, Agrobacterium tumefaciens, Azotobacter chroococcum, Azispirillum brasiliense, Bradyrhizobium arachidis, Bradyrhizobium japonicum, Bordetella pertussis, Brucella abortus, Brucella melitensis, Brucella suis, Burkholderia A39, Burkhulderia rinojensis, Campylobacter coli, Campylobacter foetus, Campylobacter jejuni, Chromobacterium subsugae, Curvibacter gracilis, Escherichia coli, Francisella tularensis, Haemophilus aphrophilus, Haemophilus haemolyticus, Haemophilus influenzae, Haemophilus parahaemolyticus, Haemophilus parainfluenza, Helicobacter pylori, Herbaspirillum hiltneri, Herbaspirillum lusitanum, Herbaspirillum rhizosphaerae, Klebsiella pneumoniae, Legionella pneumophilia, Luteibactor rhizovicina, Mesorihizobium cicero, Neisseria gonorrheae, Neisseria meningitidis, Pasteurella multocida, Pseudomonas aeruginosa, Pseudomonas azotoformans, Pseudomonas baetica, Pseudomonas brassicacearum, Pseudomonas brenneri, Pseudomonas cholororaphis, Pseudomonas fluorescens, Pseudomonas gessardii, Pseudomonas grimontii, Pseudomonas koreensis, Pseudomonas lini, Pseudomonas sp. JD18, Pseudomonas marginalis, Pseudomonas moraviensis, Pseudomonas palleroniana, Pseudomonas poae, Pseudomonas protegens, Pseudomonas psychrotolerans, Pseudomonas putida, Pseudomonas reinekei, Pseudomonas salomonii, Pseudomonas thivervalensis, Pseudomonas trivialis, Pseudomonas umsongensis, Pseudomonas viridiflava, Rhizobium azooxidifex, Rhizobium leguminosarum, Rhizobium lusitanum, Rickettsia rickettsii, Salmonella typhimurium, Serratia plymuthica, Shigella boydii, Shigella dysenteriae, Shigella flexneri, Shigella sonnei, Sinorhizobium meliloti, Sphingomonas PDD-69b-4, Sphingomonas strain A3K041, Stenotrophomonas rhizophila, Variovorax ginsengisoli, Variovorax paradoxus, Vibrio cholerae, Vibrio opticus, Yersinia enterocolitica, Proteus mirabilis, Yersinia pestis, and Yersinia pseudotuberculosis.

10. A method for increasing the storage stability of at least one Gram-negative bacteria by
(a) subjecting a wet mass of Gram-negative bacteria to spray drying; and
(b) contacting the spray-dried Gram-negative bacteria from step (a) to a mixture comprising at least one hydrophobic, partially water-insoluble polyglycerol ester in combination with at least one emulsifier to form the composition of Gram-negative bacteria,
wherein the polyglycerol ester has a general formula (I) of,
MₐD_{b}T_{c} Formula (I)
wherein, M=[C₃H₅(OR)₂O_{1/2}],
D=[C₃H₅(OR)₁O_{2/2}],
T=[C₃H_{503/2],}
a=1 to 10, preferably 2 to 3, more preferably 2;
b=0 to 10, preferably greater than 0 to 5, more preferably 1 to 3;
c=0 to 3, preferably 0 to 1, more preferably 0;
wherein, the sum total of a+b+c is 1 to 20, preferably 2 to 4, more preferably 3
wherein the radicals R are each independently selected from the group consisting of acyl radicals R′—C(=0)- and H, with the proviso that at least one radical R is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

11. The method according to claim 10, wherein the spray-dried Gram-negative bacteria of step (a) has a water activity of 0.05 to 0.4.

12. The method according to claim 10, of Gram-negative bacteria selected from the group consisting of Agrobacterium sp., Azispirillum sp. Azotobacterium sp., Bacteroides sp., Bradyrhizobium sp., Burkholderia sp., Chromobacterium sp Curvibacter sp., Fusobacterium sp., Herbaspirillum sp., Janthinobacterium sp., Luteibactor sp., Lysobacter sp., Massilia sp., Mesorhizobium sp, Mitsuaria sp., Pseudomonas sp., Paenibacillus sp., Rhizobium sp., Salmonella sp., Serratia sp., Sinorhizobium sp., Sphingomonas sp., Stenotrophomonas sp. and Variovorax sp.

13. The method according to claim 10, wherein the spray-drying in step (a) is carried out in the presence of a silica, an isomalt and/or gum arabic.

14. A liquid composition of Gram-negative bacteria comprising
a spray-dried mass of Gram-negative bacteria with an activity of water of 0.05 to 0.4;
a silica,
an isomalt; and
a mixture comprising at least one hydrophobic, partially water-insoluble polyglycerol ester in combination with at least one emulsifier
wherein the polyglycerol ester has a general formula (I) of,
MₐD_{b}T_{c} Formula (I)
wherein, M=[C₃H₅(OR)₂O_{1/2}],
D=[C₃H₅(OR)₁O_{2/2}],
T=[C₃H_{503/2],}
a=1 to 10, preferably 2 to 3, more preferably 2;
b=0 to 10, preferably greater than 0 to 5, more preferably 1 to 3;
c=0 to 3, preferably 0 to 1, more preferably 0;
wherein, the sum total of a+b+c is 1 to 20, preferably 2 to 4, more preferably 3
wherein the radicals R are each independently selected from the group consisting of acyl radicals R′—C(=0)- and H, with the proviso that at least one radical R is not equal to H;
wherein the radicals R′ are each independently selected from the group consisting of monovalent aliphatic, saturated or unsaturated hydrocarbon radicals with 3 to 39, preferably 7 to 21 more preferably with 9 to 17 carbon atoms.

15. The liquid composition of Gram-negative bacteria according to claim 14, wherein the emulsifier is a sorbitan fatty acid ester or an ethoxylated sorbitan fatty acid ester.
